SEQUENCE LISTING (8 sequences)

SEQ ID NO: 1 - DNA, 31 bp, Artificial Sequence
  N-Mer1 primer
agagcggccgcggaagtgacaaagcagaacc

SEQ ID NO: 2 - DNA, 29 bp, Artificial Sequence
  x-exon1 primer
agactcgagggatgagggaagcgggtgtg

SEQ ID NO: 3 - DNA, 21 bp, Artificial Sequence
  afp#1 primer
cccctgctctgttaattattg

SEQ ID NO: 4 - DNA, 20 bp, Artificial Sequence
  afp#2 primer
gaaaatagctcccaagtcac

SEQ ID NO: 5 - DNA, 22 bp, Artificial Sequence
  lacZ#1 primer
acaacgtcgtgactgggaaaac

SEQ ID NO: 6 - DNA, 18 bp, Artificial Sequence
  lacZ#2 primer
taatgggataggttacgt

SEQ ID NO: 7 - DNA, 2009 bp, Apodemus
  CDS (42)...(1856)
tcccacttccagcactgcctgcggtgaaggaacaagcagccatgaagtggatcacacccgcttccctcatcctcctgctacatttcgctgcgtccaaagcattgcacgaaaatgagtttgggatagcttccacgttagattcctcccagtgcgtgacggagaagaatgtgcttagcatagctaccatcacctttacccagtttgttccggaagccaccgaggaggaagtgaacaaaatgactagcgatgtgttggctgcaatgaagaaaaactctggcgatgggtgtttagaaagccagctatctgtgtttctggatgaaatttgccatgagacggaactctctaacaagtatggactctcaggctgctgcagccaaagtggagtggaaagacatcagtgtctgctggcacgcaagaagactgctccggcctctgtcccacccttccagtttccagaacctgccgagagttgcaaagcacatgaagaaaacagggcagtgttcatgaacaggttcatctatgaagtgtcaaggaggaaccccttcatgtatgccccagccattctgtccttggctgctcagtacgacaaggtcgttctggcatgctgcaaagctgacaacaaggaggagtgcttccagacaaagagagcatccattgcaaaggaattaagagaaggaagcatgttaaatgagcatgtatgttcagtgataagaaaatttggatcccgaaacctccaggcaacaaccattattaagctaagtcaaaagttaactgaagcaaattttactgagattcagaagctggccctggatgtggctcacatccacgaggagtgttgccaaggaaactcgctggagtgtctgcaggatggggaaaaagtcatgacatatatatgttctcaacaaaatattctgtcaagcaaaatagcagagtgctgcaaattacccatgatccaactaggcttctgcataattcacgcagagaatggcgtcaaacctgaaggcttatctctaaatccaagccagtttttgggagacagaaattttgcccaattttcttcagaggaaaaaatcatgttcatggcaagctttcttcatgaatactcaagaactcaccccaaccttcctgtctcagtcattctaagaattgctaaaacgtaccaggaaatattggagaagtgttcccagtctggaaatctacctggatgtcaggacaatctggaagaagaattgcataaacacatcgaggagagccaggcactgtccaagcaaagctgcgctctctaccagaccttaggagactacaaattacaaaatctgttccttattggttacacgaggaaagcccctcagctgacctcagcagagctgatcgacctcaccgggaagatggtgagcattgcctccacgtgctgccagctcagcgaggagaaatggtccggctgtggtgagggaatggccgacattttcattggacatttgtgtataaggaatgaagcaagccctgtgaactctggtatcagccactgctgcaactcttcgtattccaacaggaggctatgcatcaccagttttctgagggatgaaacctatgcccctcccccattctctgaggataaattcatcttccacaaggatctgtgccaagctcagggcaaagccctacagaccatgaaacaagagcttctcattaacctggtgaagcaaaagcctgaactgacagaggagcagctggcggctgtcactgcagatttctcgggccttttggagaagtgctgcaaagcccaggaccaggaagtctgtttcacagaagagggtccaaagttgatttccaaaactcgtgatgctttgggcgtttaaacatctccagaaggaagagtggacaaaaaaatgtgttgacgctttggtgtgagccttttggcttaactgtaactgctagtactttaaccacatggtgaagatgtccatgtgagatttctataccttaggaataaaaacttttcaactatt

SEQ ID NO: 8 - protein, 605 aa, Apodemus
MKWITPASLILLLHFAASKALHENEFGIASTLDSSQCVTEKNVLSIATITFTQFVPEATEEEVNKMTSDVLAAMKKNSGDGCLESQLSVFLDEICHETELSNKYGLSGCCSQSGVERHQCLLARKKTAPASVPPFQFPEPAESCKAHEENRAVFMNRFIYEVSRRNPFMYAPAILSLAAQYDKVVLACCKADNKEECFQTKRASIAKELREGSMLNEHVCSVIRKFGSRNLQATTIIKLSQKLTEANFTEIQKLALDVAHIHEECCQGNSLECLQDGEKVMTYICSQQNILSSKIAECCKLPMIQLGFCIIHAENGVKPEGLSLNPSQFLGDRNFAQFSSEEKIMFMASFLHEYSRTHPNLPVSVILRIAKTYQEILEKCSQSGNLPGCQDNLEEELHKHIEESQALSKQSCALYQTLGDYKLQNLFLIGYTRKAPQLTSAELIDLTGKMVSIASTCCQLSEEKWSGCGEGMADIFIGHLCIRNEASPVNSGISHCCNSSYSNRRLCITSFLRDETYAPPPFSEDKFIFHKDLCQAQGKALQTMKQELLINLVKQKPELTEEQLAAVTADFSGLLEKCCKAQDQEVCFTEEGPKLISKTRDALGV

(19) United States
(12) Patent Application Publication
(10) Pub. No.: US 2006/0234278 A1
(43) Pub. Date: Oct. 19, 2006

(54) Sequences used to identify if a female mammal comprises a mutation in the alpha-fetoprotein sequence(S) or a partial or total deletion of the alpha-fetoprotein sequence(S)

(76) Inventors: Philippe Gabant, Brussels (BE); Josiane Roscam-Szpirer, Waterloo (BE)

Correspondence Address: KNOBBE MARTENS OLSON & BEAR LLP, 2040 MAIN STREET, FOURTEENTH FLOOR, IRVINE, CA, 92614, US

(21) Appl. No.: 11/397,356

(22) Filed: Apr. 3, 2006

Related U.S. Application Data
(63) Continuation-in-part of application No. 10/031,021, filed on Mar. 19, 2002, now Pat. No. 7,022,892.
(60) Provisional application No. 60/143,269, filed on Jul. 12, 1999.

(51) Int. Cl. C12Q 1/68; A61K 31/56

(52) U.S. Cl. 435/6; 514/169

(57) ABSTRACT

The present invention is related to specific sequences, preferably present in a diagnostic kit to identify if a female mammal comprises in her genome a mutation in the alpha-fetoprotein sequence or a partial or total deletion of this alpha-fetoprotein sequence, present heterozygously or homozygously (on both allele).

### CROSS-REFERENCE TO RELATED APPLICATIONS

This application is a continuation-in-part of the U.S. application Ser. No. 10/031,021, filed Mar. 19, 2002, which is a US National Phase of International Patent Application No.: PCT/BE00/00081, filed Jul. 11, 2000, designating the US and published in English on Jan. 18, 2001 as WO 01/03501, which claims the benefit of priority of U.S. Provisional Application No. 60/143,269, filed Jul. 12, 1999, all of which are hereby expressly incorporated by reference in their entireties.

### FIELD OF THE INVENTION

The present invention is related to specific sequences (such as probes or primers), possibly present in a diagnostic kit, to identify if a female mammal comprises in her genome a mutation in the alpha-fetoprotein sequence(s) or a partial or total deletion of these alpha-fetoprotein sequence(s) present heterozygously or homozygously (on both allele).

The sequences are used in the field of diagnosis in order to identify if the female mammal is a sterile female, does not present a menstrual cyclization and/or does not allow uteral implantation of an embryo.

The sequences are also used for a diagnosis applied upon a female mammal, in order to identify if the mutation partial or total deletion is present in the genome of the mammal heterozygously or homozygously, and therefore if heterozygous female or male can transmit the mutation, the partial or the total deletion to its descendants and therefore may obtain descendants made of sterile females.

### DESCRIPTION OF THE RELATED ART

Alpha-fetoprotein (AFP) is a glycoprotein present in the serum and a classical oncofetal marker. This protein is expressed at high levels during fetal life in the liver and the visceral endoderm of the yolk sac, and at lower levels in the developing gastrointestinal tract; in the adult serum, only trace amount is detected. The protein expressed by the embryos is secreted and present in the maternal blood circulation during gestation, the level of AFP concentration in the maternal serum is use to detect fetuses with spina bifida or Down's syndrome.

The reasons for this altered AFP level associated with those pathologies are not understood, but they have been used extensively in prenatal screening. The synthesis of AFP decreases dramatically after birth and only trace amounts are detected in adult liver.

### SUMMARY OF THE INVENTION

The present invention is related to new models (animal models) as well as new methods and devices for the study, the testing and/or the screening of fertility or contraceptive methods, compounds and compositions intended for adult mammals (including humans) and/or for the study, the testing and/or the screening of new methods, compounds or compositions intended for the treatment and/or the prevention of osteoporosis.

The present invention is related to new diagnostic tools, especially specific sequences, such as probes or primers that could be used to identify the following phenotypes in a mammal female (including the human female):
if the female mammal is sterile,
if the female mammal does not present a menstrual cyclization,
if the female mammal does not allow uteral implantation of a embryo and/or,
if the female mammal could present a predisposition to said phenotype(s) and may transmit or could transmit said phenotype(s) to her descendants.

The present invention is also related to a method for diagnosing if a mammal female (including the human female) presents the above mentioned phenotypes due to a mutation, a partial mutation (AFP DNA sequence) in the mammal female genome (including the human female genome).

The present invention is further related to such tools which allow to characterize which genetic modifications in the AFP sequence will induce one or more of these phenotypes.

The present invention is also related to a method for the prevention of one or more of these phenotypes in a mammal female by a treatment of her mother female mammal carrying descendant(s) which may present such genetic modification.

The present invention is related to a non-human genetically modified mammal (preferably a knock-out mouse) comprising a mutation, a partial or total deletion in a genetic sequence encoding a mammal alpha-fetoprotein (AFP) described in GenBank (v00743).

The inventors have demonstrated that a mouse female presenting modifications in the genetic sequence encoding the mammal alpha-fetoprotein (AFP) especially a total deletion of this genetic sequence presented homozygously on both allele, presents a specific phenotype such as: female sterility, no menstrual cyclization and no uteral implantation of an embryo.

Furthermore, Sharony et al. (2004 Eur. J. Human Genet. 12:871-874) have studied two families with congenital AFP deficiency and searched for mutations in the AFP gene. The authors have identified one mutation of two base deletions in exon 8, in both families that leads to congenital deficiency of AFP. All the affected children were found to be homozygous for the mutation as was one of the fathers. The authors also describe that the affected individuals were asymptomatic and presented normal development. Therefore, the identification of this mutation in the AFP gene demonstrates for the first time that deficiency of AFP is compatible with human normal fetal development and further reproduction in males.

This publication also demonstrates that the induced genetic modification observed in a mouse may also affect the human population but does not modify a human development.

However, these authors have not yet demonstrated that such genetic modification in the AFP genetic sequence may lead to female sterility.

The present invention is related to a method for diagnosing if a mammal female (including a human female) presents a specific phenotype being female sterility, no menstrual cyclization and/or no uteral implantation of an embryo or may present a predisposition to have one or more of these phenotypes or present a predisposition to transmit these phenotypes to her descendant(s).

This method of diagnosis comprises the step of detecting in the mammal female genome (including the female human genome) a mutation, a partial or total deletion of the genetic sequence encoding the mammal alpha-fetoprotein (AFP) sequence.

A mutation or deletion in the AFP genetic sequence means a mutation or a deletion which affects the structure and the activity of the AFP gene product, especially the binding affinity of the AFP protein to estrogen.

Preferably, the portion of the alpha-fetoprotein that comprises this (these) mutation(s) or deletion(s), is the AFP domain III made of about 200 amino acids (as described by Festin (1999)).

Said mutation/deletion could be also present in the C terminal extremity of the corresponding AFP amino acids.

Preferably, the method for diagnosing is based upon the step of comparing the genome of the female mammal subject (including a human female patient) to a genome of another mammal (of the same species) which does not present the mentioned phenotype or does not present a predisposition to present this phenotype.

Preferably, said method is obtained by contacting a cell sample containing a target nucleic acid molecule obtained from this mammal with a complementary capture nucleic acid probe which hybridizes under stringent conditions this target nucleic acid sequence encoding the mammal alpha-fetoprotein AFP or a portion thereof; the said capture nucleic acid probe differing from a mammal alpha-fetoprotein (AFP) wild type sequence by at least one nucleotide.

Furthermore, said method comprises the step of detecting a signal resulting from this hybridization (by complementary base pairing) and/or the step of characterizing the presence of one or more mutation in the target alpha-fetoprotein nucleotide sequence by methods well known by the person skilled in the art (by sequencing the full sequence of AFP genetic sequence and identifying if said genetic sequence comprises one or more mutation(s), partial or total deletion (including any modification which may result in non-expression or not normal expression of the AFP protein.

Preferably, the capture nucleic acid probe is bound upon a solid support, preferably according to a microarray.

Another aspect of the present invention concerns a diagnosis method comprising the step of performing a genetic amplification of target sequences encoding a mammal alpha-fetoprotein (AFP) and present in the genome of a cell of the female mammal subject (including the female human patient), preferably by specific sequences (primers) which are able to hybridize to complementary sequences present in the genome of the mammal subject and able to amplify only said target alpha-fetoprotein (AFP) genetic sequence, or portions thereof, and the step of identifying if the amplified target sequences present one or more mutation(s), partial or total deletion(s).

The method according to the invention could be advantageously combined with other methods used to characterize the genotype of sterile mammal female subjects, especially human female patients.

Preferably, said method for the characterization of the genotype of a sterile female mammal subject is provided for targeting mutation, partial or total deletion affecting other genes, involved in the same phenotype(s) (sterility, absence of menstrual cyclization, absence of uteral implantation of a embryo).

Another aspect of the present invention concerns tools for performing said method, especially specific sequences (such as primers or probes), which are able to hybridize to complementary target nucleotide sequences that are present in the genome of a female mammal subject. The corresponding target sequence is a sequence that may comprise a mutation, a partial or total deletion of the alpha-fetoprotein DNA sequence, such genetic modification (mutation, partial or total deletion) could be present in the genome of the mammal, heterozygously or homozygously.

Such specific sequences are also used to identify if said mutation, partial or total deletion of the AFP DNA sequence in the genome of a female mammal subject results in the specific phenotype (female sterility, no menstrual cyclization and/or no uteral implantation of an embryo).

This specific (capture) sequence (primer or probe) is preferably bound upon a solid support surface, preferably according to a microarray. Therefore, a further aspect of the present invention concerns a solid support which comprises these specific (capture) sequences bound according to a microarray (biochips).

Preferably, at each location of the microarray, are bound specific sequences which differ between them by at least one nucleotide and which are used to hybridize to complementary target sequences to be detected and that could be present in the genome of the female mammal subject.

Preferably, the sequences possibly bound upon the solid support (such as a microarray), are present in a diagnostic kit which comprises other elements, means and media for performing such detection and/or identification.

Those means and media are for example means and media used for a genetic amplification (by PCR, LCR, NASBA, etc), or for a specific hybridization between complementary nucleotide sequences and/or label(s) possibly bound directly or indirectly to the sequences according to the invention.

The label could be any suitable molecule which could generate a signal that could be used in the detection and/or identification method according to the invention.

Preferably, this label comprises molecules allowing detection by fluorescence, chemiluminescence, bioluminescence, colorimetry or are radioactive labels.

Preferably, the sequences are present in a diagnostic kit which comprises other elements (means and media) for performing such identification.

Such means and media are for example means and media (buffers, tag polymerase, . . . ) used for a genetic amplification (by PCR-LCR, NASBA, etc), and/or label(s) possibly bound directly or indirectly to the sequences according to the invention.

The diagnostic kit or microarray according to the invention could also comprise other sequences dedicated to other genetic modifications involved in sterility phenotype of a subject mammal female.

Another aspect of the present invention is related to a method of treatment and/or prevention of the above mentioned phenotype into a female mammal subject, especially a female human patient; said method comprising the steps of:
identifying if a mammal female subject presents a mutation, partial or total deletion of the alpha-fetoprotein DNA sequence in her genome and may transmit this mutation, partial or total deletion to her descendant(s), especially to her daughter which may present such mutation, partial or total deletion homozygously and associated with the above described phenotype;
treating the female mammal subject patient, in order to obtain the birth of a female mammal which does not present the phenotype associated with mutation, partial or total deletion of the AFP DNA sequence.

Preferably, this treatment is a hormonal treatment (by administration of aromatase inhibitors) applied during the gestation of the child, to restore fertility, to recreate a menstrual cyclization of the female and/or to allow uteral implantation of the embryo or after the birth of the female mammal subject descendant.

Another aspect of the present invention is related to a non-human mammal pluripotential embryonic stem cell, preferably a mouse pluripotential embryonic stem cell comprising a partial or total deletion of a genetic sequence encoding a mammal AFP. Said stem cell can be advantageously used to obtain the non-human genetically modified mammal according to the invention by methods well known by the person skilled in the art described hereafter.

A further aspect of the present invention is related to the use of the non-human mammal according to the invention for the study, the testing and/or the screening of known or unknown anti-neurodegenerative, anti-osteoporosis, fertility and/or contraceptive methods, compounds or compositions.

Preferably, the portion of the alpha feto-protein that comprises this mutation or partial deletion is the AFP domain III made of about 200 amino acids, as described by Festin (51). This mutation or deletion could also be present in the C terminal extremity of the alpha fetoprotein amino acid sequences.

A last aspect of the present invention is related to study, testing and/or screening methods and devices comprising the AFP or a portion of said AFP, preferably the AFP domain III comprising about 200 amino acids (as described by Festin (51), being fixed upon a solid support and used as a substrate for known or unknown target compounds or compositions in a competitive test or method. Said device comprises also a medium comprising (possibly labeled) estrogens.

The device according to the invention can be a chromatographic column upon which the AFP or the portion thereof is fixed or a study, testing and/or screening kit comprising disposed separately the various media necessary for said study, testing and/or screening.

According to a preferred embodiment of the present invention, said device or kit may comprise a cell having integrated an estrogen-sensitive (prolactin) promoter gene whose activation may result from the fixation of known or unknown target compounds or compositions upon the AFP. Said known or unknown target compound or composition could be used advantageously as an agonist of an estrogen.

The present invention is also related to this unknown target molecule (agonist, antagonist or partial agonist) of estrogens screened and identified by the method and the device according to the invention. This unknown target molecule finds application in the field of fertility and/or contraceptive methods and can be included in compositions and/or used for the treatment and/or the prevention of fertility and/or neurodegenerative disease, osteoporosis.

### BRIEF DESCRIPTION OF THE DRAWINGS

FIG. 1A illustrates the targeted disruption of the afp gene to generate alleles which are deleted for most of the sequence of exon 1, for exon 2 and for exon 3. FIG. 1B illustrates the detection of homologous insertion by Southern blot analysis. FIG. 1C illustrates a Southern blot analysis of the progeny of homozygous (−/−) chimeric animals.

FIG. 2A is a Northern blot analysis of total RNA from embryonic liver. FIG. 2B is a Western blot analysis of embryonic liver and amniotic fluid protein extracts.

FIGS. 3A to 3H illustrate expression of the lacZ gene in embryonic and adult tissue. FIGS. 3A, 3G and 3H illustrate expression in adult liver and gut cells. FIGS. 3B to 3F illustrate expression of the lacZ gene in embryonic tissues.

FIG. 4A illustrates the structure of the ovary (arrow) and uterus of an adult afplacZ1/lacZ1(−/−) female. FIG. 4B illustrates the ovary from a 12 week old afplacZ1/lacZ1 animal. FIG. 4C illustrates the ovary from a 12 week old mutated afplacZ1/lacZ1 female. FIG. 4D illustrates the general histological structure of afplacZ1/lacZ ovaries. FIG. 4E illustrates the structure of wild type ovaries at 4 months.

FIG. 5 represents a schematic view of the GnRH pathway. Genes the expression of which is affected in the AFP KO. Females are indicated in bold. Fold changes in mRNA levels are shown as ratios AFP KO:WT.

FIG. 6A-E represents relative expression levels of genes in the pituitary (Isp2 (A), Ucp1 (B), Egr1 (C), Gnrhr (D)) and the hypothalamus (Gnrh2 (E)). Values are normalized with the Hprt gene values. WT: wild type allele homozygous females (n=16); HET: heterozygous females (n=9); KO: homozygous AFP KO females (n=7); KO ATD: prenatally ATD-treated AFP KO females (n=7).

### DETAILED DESCRIPTION OF THE PREFERRED EMBODIMENT

### Example 1

### Generation of Mice Carrying a Germ-Line Mutation in the AFP Gene

A clone containing a 129 genomic fragment of AFP loci was isolated from a lambda library. The library was screened with a probe containing the mouse afp promoter. The genomic insert of about 16 kb was subcloned in pKIL-PCR2 (Gabant et al., 20). The targeting vector (pAFP K.O-1), consist of two recombination arms. The 5′ arms (2.5 kb) were generated by polymerase chain reaction (PCR) using the following primers: N-Mer1: agagcggccgcggaagtgacaaagcagaacc (SEQ ID NO: 1) annealing to the MerI sequence of the afp enhancer 1 (Godbout et al. (48, 49) and a primer of the X-exon1: agactcgagggatgagggaagcgggtgtg (SEQ ID NO: 2) complementary to the afp exon1. The PCR fragment generated using Pfu polymerase (Stratagene) was cloned in the pCR-blunt vector (Invitrogen).

The 3′ arms were subcloned from the lambda into pBSIIKS+ vector (Stratagene). The 5′ recombination arm was introduced upstream the 3′ recombination arm. The IRES lacZ/neo reporter-selective cassette was introduced between these recombination arms. The tk2 negative selective marker was introduced into the SalI site to generate pAFP KO-1. This construction was linearized with NotI and electroporated into E14 ES cells. Correctly targeted clones were identified by Southern blot analysis using an external probe from the 5′ region.

Es Cell Injections and Animal Genotyping

Recombinant ES cells carrying the targeted allele were injected in C57BL/6J blastocysts. Animals were genotyped by extraction of DNA from tails.

RNA isolation, Northern Blot Analysis

Total RNA was isolated using Trizol (Gibco BRL) extraction according to the manufacturer instructions. For the Northern analysis 20 μg of total RNA were electrophoresed and transferred to nylon membranes as described. Filters were then hybridized.

Western Blot Analysis

Proteins were separated by SDS-PAGE using 7.5% polyacrylamide gels in a Bio-Rad Mini Protean gel chamber and blotted onto Nitrocellulose filters in a Bio-Rad Trans Blot chamber according to the manufacturer's instructions. Proteins were detected using anti-AFP, anti-Albumin; anti Betagalactosidase serum (ICN Biochemicals) the signal was detected with ECL detection system (Amersham).

LacZ Reporter Gene Expression

To isolate embryonic stages, natural matings were set up and presence of a vaginal plug at noon the following day was taken as 0.5 days of gestation. Staged embryos were stained with X-Gal as wholemounts (PNAS USA 93:1677-1682). For cryostat sectioning, tissues were embedded in optimal cutting temperature (OTC) compounds (Miles, Inc., Elkart, Ind.), and sections stained for X-Gal were counterstained with hematoxylin and eosin, and mounted.

Targeted Mutagenesis of the afp Gene

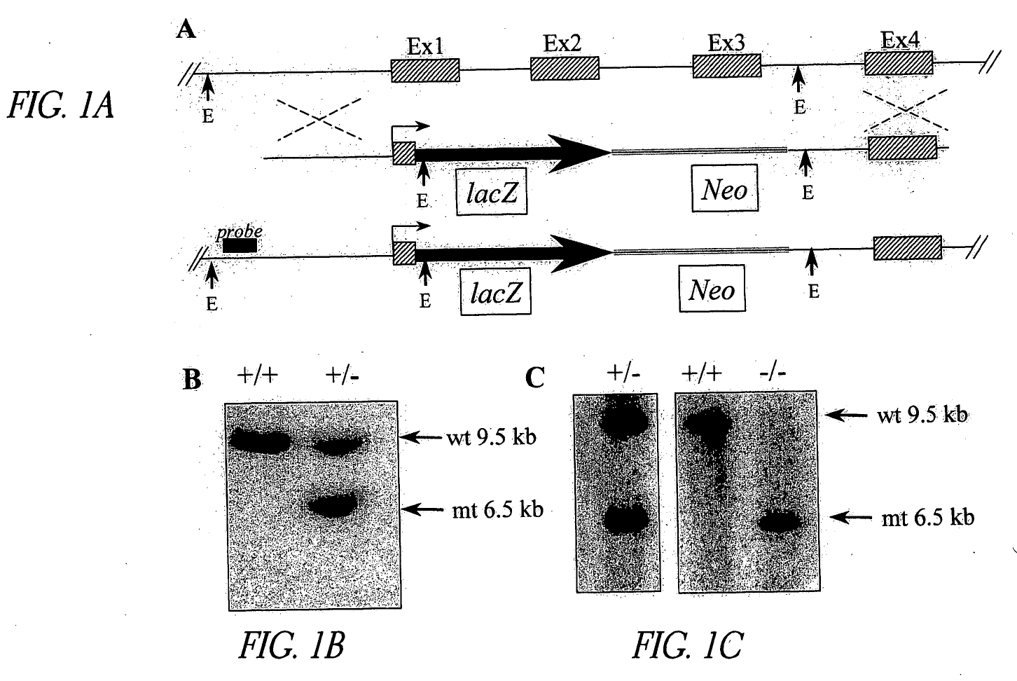

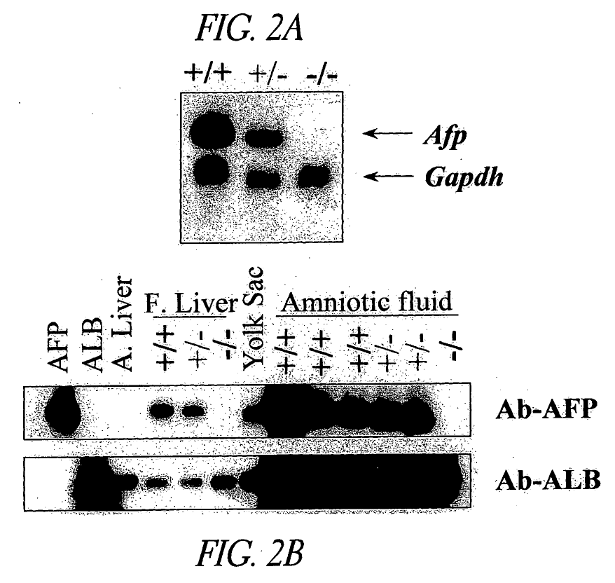

The afp gene was disrupted by gene targeting in embryonic stem (ES) cells. The lacZ reporter was introduced in afp gene by homologous recombination and placed under the control of the AFP promoter-enhancer region. The resulting allele is deleted for most of the sequence of exon1, for exon2 and 3 (FIG. 1A) and homologous insertion was detected by Southern analysis (FIG. 1B). To test the functionality of the reporter one may take advantage of the observation that AFP is expressed in embryoid bodies (Abe et al., 58). Reporter gene activity is highly turned on in some cells of these bodies (FIG. 2A). No expression of the reporter was detected in undifferentiated ES cells grown in the presence of LIF.

ES cells afp^{lacZ1/+} were injected into C57BL/6J blastocysts. Chimeric animals were obtained and mated with outbred CD1 or inbred 129/CGR to test for germ line transmission. Phenotypically normal heterozygous mice afp^{lacZ1/+} were generated and detected by Southern blot (FIG. 1C).

Reporter Expression Analysis

Expression of the lacZ reporter gene expression in embryonic and adult tissues was analyzed. As shown in FIG. 2 the β-galactosidase activity was detected in predicted embryonic tissues. In the visceral endoderm only patches of cells were observed to turn the reporter strongly on. In the adult tissues tested specific staining was only detected in cells of the liver and in cells of the gut.

Animals without AFP are Viable

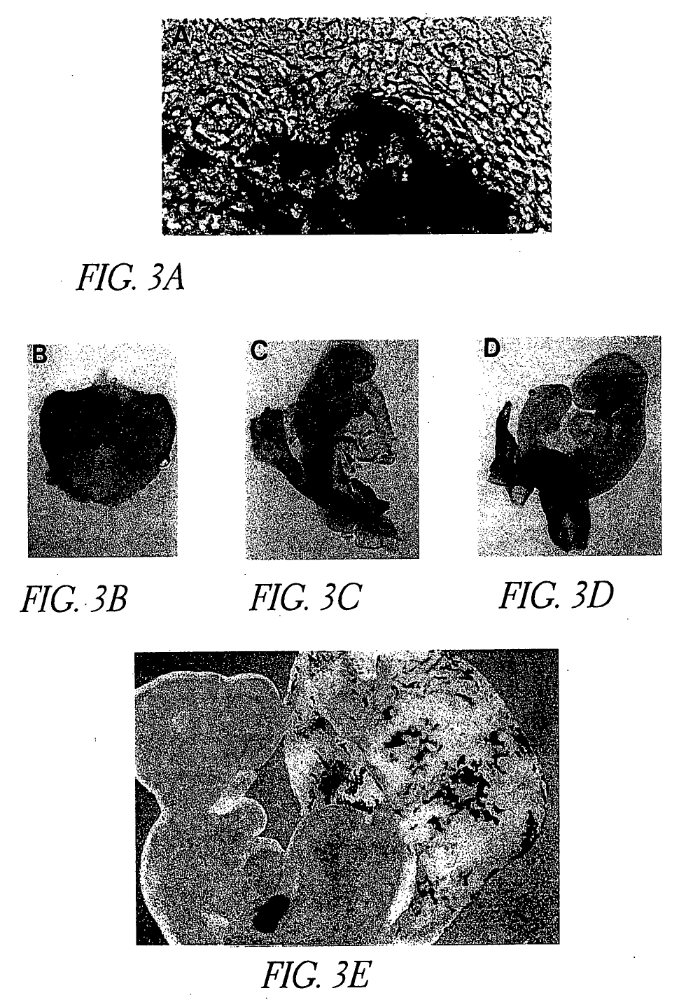

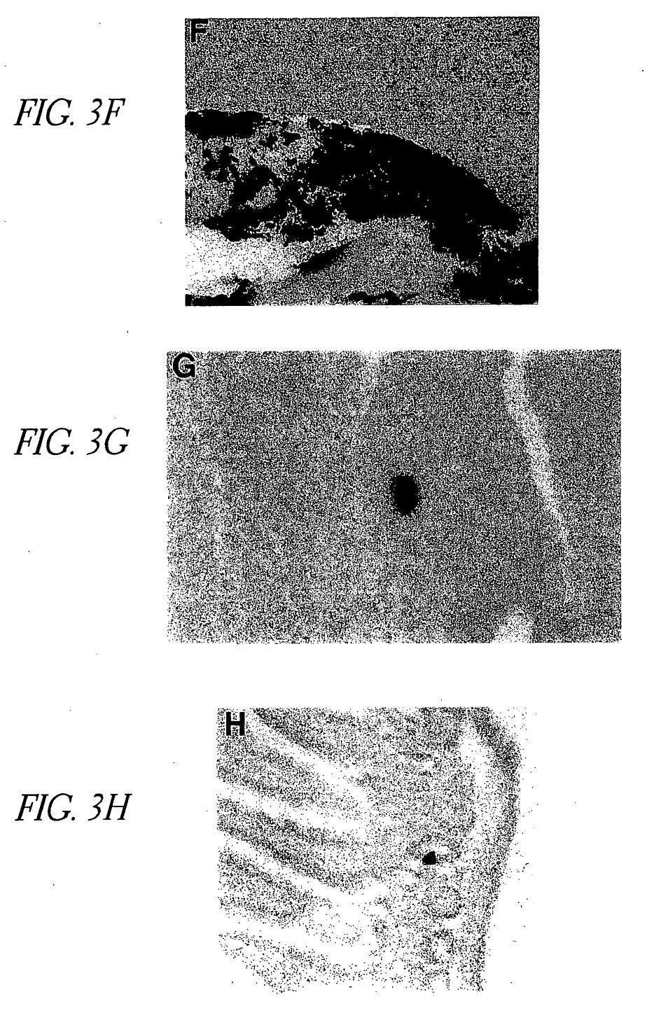

Intercrosses of heterozygotes (afp^{lacZ1/+}) gave rise to viable, apparently normal homozygous mutant mice at a Mendelian ratio in CD1 and C57/B16. On the other hand, a significant divergence was observed in the 129 background (Table 1). To determine whether the targeted allele indeed results in a null mutation, total RNA from the liver of embryos was analyzed by Northern blot hybridization (FIG. 3A). A strong signal at 2.2 kb corresponding to the afp transcript was detected in wild type and heterozygous embryos. No signal was detected in RNA samples extracted from homozygous embryonic liver, showing that the recombination disrupted the afp transcript in these animals. A Western blot was also performed on embryonic liver and amniotic fluid protein extracts (FIG. 3B) and a strong signal was detected with the wild type extracts while no band corresponding to AFP was visible in the homozygous extracts demonstrating that these animals do not express AFP.

TABLE 1
Intercrosses

| | |
|---|---|
| Parents | Offsprings |

| Strains | Male | Female | +/+ | +/− | −/− |
|---|---|---|---|---|---|
| CD1 | +/− | +/− | 108 | 258 | 102 |
| 129 | +/− | +/− | 34 | 48 | 13 |
| C57/black-6 | +/− | +/− | 19 | 43 | 19 |

AFP is Required for Female Fertility

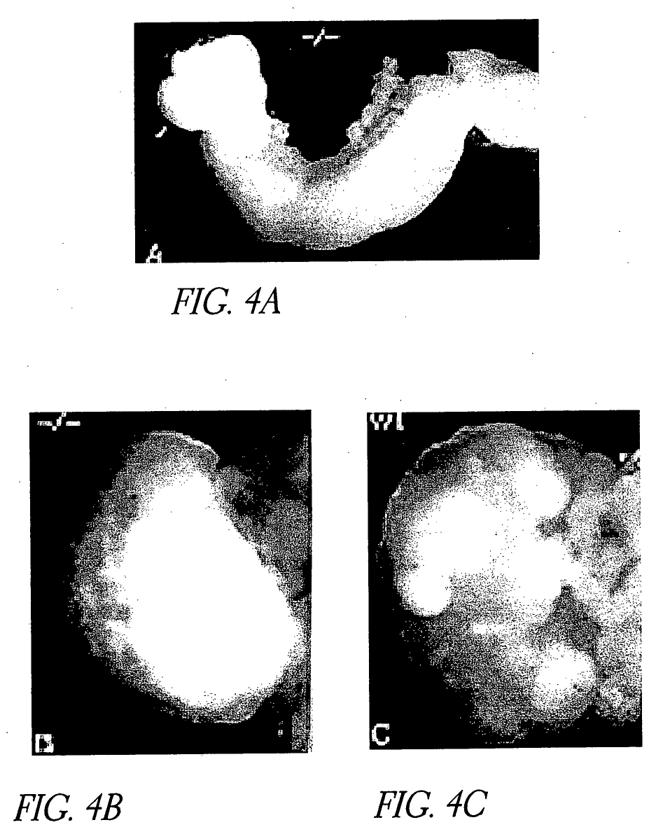

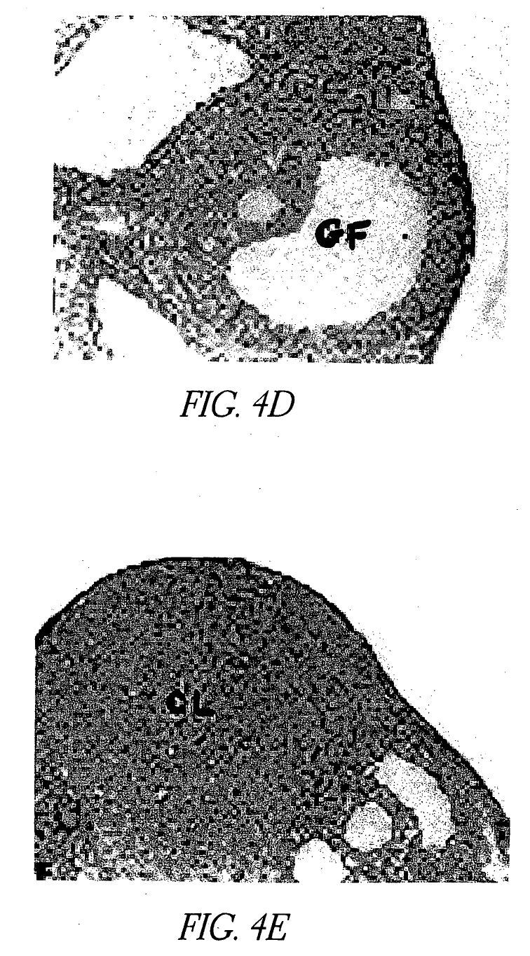

The Mendelian ratio obtained in CD1 and C57/black-6 background demonstrates that there is no reduction in the intercrosses. On the other hand, the divergence observed in 129 background suggests that AFP is involved in the gestation and that its importance is only revealed in some genetic contexts. In these litters derived from intercrossing heterozygous animals, homozygous embryos develop in the presence of their wild type and heterozygote littermates. AFP produced by these embryos is secreted and present in the maternal serum. To determine whether afp^{lacZ1/lacZ1} mice are able to develop in the complete absence of AFP, afp^{lacZ1/lacZ1} males and afp^{lacZ1/lacZ1} females were mated. No pups were obtained from these intercrosses suggesting an essential role of AFP for development and/or fertility (see Table 2). To test if fertility was affected, afp^{lacZ1/lacZ1} males and females were mated with wild type animals. Males homozygous for an afp disrupted allele appeared fertile and sired offspring but homozygous females never produce any live offspring. To test if natural matings occur with the afp^{lacZ1/lacZ1} females leave with wild type males the vaginal plug were checked. No plugs were detected with those females, showing that the origin of the observed infertility is due to an absence of mating. To identify the defect underlying the reproductive capacity of homozygous females, the reproductive system of those animals was analyzed. The reproductive system of the afp^{lacZ1/lacZ1} females was dissected and at this stage of the analysis appeared complete. A major anatomical difference is notable between ovaries from afp^{lacZ1/lacZ1} and afp^{+/+}: the ovaries of afp^{lacZ1/lacZ1} are smooth, this observation suggests that those females do not ovulate. Histological analysis of mature afp^{lacZ1/lacZ1} ovaries shown that their homozygous tissues do not contain corpus lutea, the lack of these structure is indicative of the absence of ovulation (see FIG. 4). The afp^{lacZ1/lacZ1} ovaries contain follicles at the different stages of maturation, this suggests that the default of AFP during the development has no effect on the female gametogenesis. However, the presence of follicles does not prove that these gametes are competent for maturation. To test the competence of the afp^{lacZ1/lacZ1} follicles, they were dissected out and analyzed their potential of maturation in vitro. Complete maturation was obtained in vitro with the dissected oocytes from afp^{lacZ1/lacZ1} animals. Taken together these data indicates that those females do not ovulate properly and thus that a signal needed to trigger ovulation is absent in the afp^{lacZ1/lacZ1} mice.

TABLE 2
Phenotypical analysis

| | |
|---|---|
| Parents | Offsprings |

| Male | Female | +/+ | +/− | −/− |
|---|---|---|---|---|
| −/− | −/− (2) | 0 | 0 | 0 |
| −/− | +/+ (6) | 0 | 71 | 0 |
| +/+ | −/− (13) | 0 | 0 | 0 |

Table 2: Infertility phenotype of the afp^{lacZ1/lacZ1} (−/−) homozygous animals were mated, no offsprings were obtained from these matings. To test fertility of the afp^{lacZ1/lacZ1} (−/−) males and females, homozygous males and females were mated with wild type (+/+) animals. For the different breedings the number of mating is given in brackets.

It was also observed that afp^{lacZ1/lacZ1} follicles are able to mature normally in vitro suggesting that the defect could be in a signal required to trigger ovulation and indeed ovulation can be induced in these animals by a superovulation protocol (Table 3).

TABLE 3
Ovulation induction in afp^{lacZ1/+} and afp^{lacZ1/lacZ1} females

| | Number of |
|---|---|
| Mice injected | oocytes obtained |
| afp^{lacZ1/+} females (9 weeks) | 37 |
| afp^{lacZ1/lacZ1} females (9 weeks) | 31 |

Table 3: Induction of ovulation in afp^{lacZ1/lacZ1} females. afp^{lacZ1/lacZ1} and afp^{lacZ1/+} females were hormonally treated to induce ovulation. The average number of postovulation oocytes obtained from 6 individual females tested.

Mouse Genotyping by PCR

In order to identify a possible mutation or deletion in the afp gene (see sequence in WO 01/03501), a specific genotyping of afp −/− mice by PCR has been developed. Two primers are used for the first afp# 1 anneals in the afp promoter region (−116 bp to −137 bp): according to the +1 of the mouse afp gene). The second primer afp#2 is complementary to the first exon of the mouse afp gene (+141 bp to +160 bp: according to the +1 of the mouse afp gene): sequence deleted in the afp −/− knock-out mouse described in the text.

Sequence afp# 1: cccctgctctgttaattattg (SEQ ID NO: 3)

Sequence afp#2: gaaaatagctcccaagtcac (SEQ ID NO: 4)

No amplification product (300 bp) was observed in afp −/− animals. This amplification is present in wild type and heterozygous mice.

To differentiate +/− from wild type, a second PCR is performed using two primers giving an amplification on the DNA introduced in the genome of the transgenic animals (this sequence is not present in wild type animals).

For the knock-out mouse for afp described in the text: a couple of primers complementary to lacZ (E. coli gene) can be used.

| | | |
|---|---|---|
| lacZ#1: | acaacgtcgtgactgggaaaac | (SEQ ID NO:5) |
| lacZ#2: | taatgggataggttacgt | (SEQ ID NO:6) |

Those primers will only give a signal (287 bp) on +/− and no signal on wild type DNA samples.

The physiological role of the alpha-fetoprotein, the most abundant serum protein expressed by mammalian embryos remains to be established. This protein related to albumin excreted by the embryos into the maternal blood circulation has attracted attention and due to its abundance it has been postulated that the presence of this protein was essential for embryonic development. To determine the function of AFP this gene was disrupted by homologous recombination in embryonic stem cells. Surprisingly homozygous afp^{lacZ1/lacZ1} are viable showing that expression of AFP by the embryo itself is not require for normal and complete development. This phenotype shows that the structure of the homozygous females was generally maintained. The different stages of ovocytes maturation are founded in these ovaries and shown to accomplish their maturation in vitro. Ovulation was induced in these females by hormonal induction and ovocytes were produced. Although AFP is synthesized at a high level during fetal life (mainly by the liver and the visceral endoderm of the yolk sac), low level of AFP mRNA has been reported in different other fetal and adult tissues as well as in adult rats. However, the level of expression in such tissues is very low. Another explanation is that the ovulation in those females is affected by the lack of AFP during development.

The fact that in adults afp^{lacZ1/lacZ1} ovulation can be induced argues for the absence in those females of a signal needed to trigger ovulation. This suggests that AFP is involved in the transportation of an element needed for ovulation in the adults.

By the disruption of the mouse AFP, one may show that fetal serum protein is required for female's ovulation.

The fact that analbuminic rats are fertile shows that at least albumin cannot rescue AFP. This show that albumin and AFP plays two different roles and that AFP is involved in the function of females ovaries.

The specific phenotype of the non-human mammals according to the invention is also illustrated in the enclosed FIG. 4, which presents the anatomical and histological analysis for the afp^{lacZ1/lacZ1} ovaries:
A: Structure of the ovary (arrow) and uterus of an adult afp^{lacZ1/lacZ1} (−/−) female;
B: Ovary from 12 weeks old afp^{lacZ1/lacZ1};
C: Ovary from a 12 weeks old wild type female: the surface distortions are due to the presence of large type follicles, whereas afp^{lacZ1/lacZ1} ovaries are smooth;
D: The general histological structure of the afp^{lacZ1/lacZ1} ovaries is not affected and mature Graafian follicles are present in those tissues (section from a fourth month old female);
E: At 4 months, the wild type ovaries exhibit large corpus lutea, indicative of successful ovulation. Those structures are never found in afp^{lacZ1/lacZ1} ovaries.

Therefore, the non-human female mammals that present homozygously said mutation, partial or total deletion in the afp gene do not cycle. The surface of the ovaries is smooth and is not characterized by the presence of large follicles. Their histological structure is not generally affected, and Graafian follicles are identified in the tissues but no large corpus lutea is present.

Furthermore, females comprising a homozygous mutation or partial or total deletion in the afp gene do not allow uterus implantation of an embryo.

Additional experiments have shown that the afp gene is not essential for the survival of the mice. Indeed, as females give birth to several animals, it was possible that the afp^{-/-} may survive to brothers and sisters (afp^{+/+} or afp^{+/-}) simultaneously present in the uterus of the female.

Therefore, in order to extrapolate the observed phenotype and genotype to the human population, the inventors have shown that blastocysts implanted one by one in pseudopregnant females will obtain the birth of alive afp^{-/-} animals that present the same phenotype as above-described (fertile males, sterile females).

Therefore, the afp^{-/-} phenotype corresponds to alive sterile females, which is a phenotype that may exist in the mouse population as well as in the human population.

### Example 2

### Gene Expression Profile in the AFP KO Female Hypothalamus-Pituitary Axis

Microarray Study

AFP KO mice are the ones described in the Example 1. The CD 1 animals used in the microarray analysis were bearing the Afp tm2Ibmm allele (knock-out, described in reference 20) and were from the 5th backcross generation. Mice were housed under a 12 h light/dark cycle (lights off at 6 h30 pm). Food and Water (Scientific Animal Food and Engineering, Augy, France) were available ad libitum. Animals were killed by cervical dislocation between the age of 4 and 5 months. Females were in the metoestrus II/dioestrus phases of the estrous cycle as established by vaginal smears, according to previous observation that AFP KO females remain in these phases (20). The pituitaries were dissected in one piece while a 4 mm side cubic piece was cut around the pituitary stalk in the brain, enabling the dissection of a zone containing the hypothalamus. Tissues were immediately processed after dissection by disruption in Dounce Homogenizer in the presence of Trizol reagent (Life Technologies, Inc. Carlsbad, Calif.). Total RNA was further extracted according to the Trizol's manufacturer's instructions and resuspended in the RNA Storage Solution (Ambion Inc, Texas, USA). RNA Preparation and Affymetrix GeneChip Hybridization.

The quality of the RNAs was checked with the Agilent 2100 Bioanalyzer (Agilent Technologies, Palo Alto, Calif.). All the RNAs used in the experiment met the quality criteria defined by Agilent. Genes expressed in each sample were analyzed on a high-density oligonucleotide microarray (MOE-430A; Affymetrix, Santa Clara, Calif.) containing 22,690 transcripts. Target preparation and microarray processing procedures were performed as described in the Affymetrix GeneChip Expression Analysis Manual. Briefly, 3 μg of total RNA were used to synthesize double-strand cDNA with SuperScript II reverse transcriptase (Life Technologies, Inc. Rockville, Md.) and a T7-(dT)24 primer (Proligo, Paris, France). Then, biotinylated cRNA was synthesized from the double-stranded cDNA using the RNA Transcript Labeling kit (Enzo Life Sciences, Farmingdale, N.Y.) and was purified and fragmented. The fragmented cRNA was hybridized into the oligonucleotide microarray, which was washed and stained with streptavidin-phycoerythrin. Scanning was performed with an Agilent Microarray Scanner.

Data Analysis

All the ‘dat’ files (corresponding to the scan image) and the ‘rpt’ files corresponding to each Genes Chip were first checked for the control of hybridization defined by the Affymetrix GeneChip Manual (uniformity of the signal, good position of the grid, scale factor, background, percent of presence, presence of house keeping genes and spikes control).

All the experiments were also analyzed with the ‘deg’ algorithm associated to the Bioconductor 1.3 software (bioconductor.org website). This algorithm analyses the quality of the mRNA by comparing the signal intensity in the 5′ and in the 3′ end of the mRNA, which should be similar if no RNA degradation occurred.

All these quality controls were positive and are available in the enclosed Table 4 (Miame Total, minimal information about a microarray experiment), available as supporting information.

The inventors also conducted an analysis using the RMA (‘robust microarray analysis’), which first calculates the probe specific correction of the PM (perfect match) probes using a model based on observed intensity being the sum of signal and noise, and then normalize the PM probes using the quantile normalization. The expression measure was calculated using a median polish (23, 24).

The ‘Cel’ files of each experiment were collected with the Bioconductor 1.3 software and treated according to the RMA package (we used the ‘quantile’ normalization). All the data were then collected in the BRB 3.1 software developed by Dr Richard Simon and Dr Amy Peng Lam 2 (linus.nci.nih.gov/BRB-ArrayTools.html website). A list of genes was generated using the Class Comparison Tool associated with BRB 3.1. Because of the small sample size, a randomized variance t-test was used with a threshold level of significance p<0.05. In addition, a FDR (False 5 Discovery Rate) correction procedure was applied using a p=0.05 (4, 5).

Real Time PCR Analysis

Results obtained by the microarray technology were confirmed using quantitative RT-PCR technology. Results from the pituitary were confirmed using the 7900HT Micro Fluidic Card (Applied Biosystems, Foster City, Calif., U.S.A) technology. For each group of animals (wild type and knock-out females) two mRNA samples that were previously analyzed using the microarray technology and an additional one not analyzed before. 1 μg of RNA of each animal was reverse transcribed using the High Capacity cDNA Archive Kit (Applied Biosystems) in a total volume of 10 μl. Then 1 μl of the RT product (100 ng cDNA) was loaded into each well of the 7900HT Micro Fluidic Card (Applied Biosystems) for quantitative PCR amplification using the Universal PCR Master Mix (Applied Biosystems). Data were analyzed using the relative quantification software available on the machine. The housekeeping gene Hprt (hypoxanthine guanine phosphoribosyl transferase) was used as reference gene for normalization. In the case of the Gnrh2 gene (gonadotropin-releasing hormone), the differential expression between normal and knock-out females (n=7 for each genotype) was tested on the 7300 Real Time PCR System (Applied Biosystems) with the absolute quantification software. 1 μg of total RNA was reverse transcribed using the High Capacity cDNA Archive Kit (Applied Biosystems) and 100 ng of cDNA was brought in the PCR reaction. The housekeeping gene Hprt was used as reference gene for normalization. PCR was performed using the qPCR MasterMix for SYBR Green I (Eurogentec, Seraing, Belgium).

Aromatase Inhibitor Treatment

Females heterozygous for the Afp tm2Ibmm allele (CD1 strain, 8th backcross level) were mated with Afp tm2Ibmm homozygous males and housed under the conditions described above. They then received a daily subcutaneous injection in the neck of 4 mg of the aromatase inhibitor ATD (1, 4, 6-androstatrien-3,17-dione, Steraloids, Newport, R.I.) dissolved in propylene glycol, from the 13.5th day of gestation (day 0.5 is defined as the day of vaginal plug detection) until the end of gestation. Controls were injected with propylene glycol only. Pups were born naturally or retrieved by Caesarian operation on embryonic day 20.5. Blood from the mothers was taken from the hart and estradiol levels were measured by classical RIA assay. Fertility of female pups was tested at the age of 2-3 months. In parallel, sisters of the female pups tested were dissected at the age of 3 months and total RNA was extracted from the pituitary and the hypothalamus as described above. cDNA was obtained using the TaqMan Reverse Transcription Reagents kit (Applied Biosystems) and real time PCR was performed on the 7300 Real Time PCR System (Applied Biosystems) with the Platinum Quantitative PCR SuperMix-UDG kit (Invitrogen) and the TaqMan Gene Expression Assays (Applied Biosystems) for pituitary genes except for Egr1. The Gnrh2 and Egr1 transcripts were quantified using the same machine and software but with the Platinium SYBR Green qPCR SuperMix UDG (Invitrogen, Carlsbad, Calif.) for PCR reactions. Statistical testing for pairwise comparison between groups was made with the t-test (alpha=0.05) or with the Mann-Whitney Rank Sum Test.

Results

Gene Expression Profile in the AFP KO Female Hypothalamus-Pituitary Axis

Total RNA from the pituitary of phenotypically normal, wild type mice (WT) and AFP KO mice (n=3 for each group) were analyzed using the Affymetrix Microarray technology on the Mouse expression set 430A. The WT females were compared to the AFP KO females. In order to reduce the background generated by the genes whose expression level was very close to the median of the expression level. Data were filtered by choosing the genes showing a variance of the log-ratio significantly different from the median of all the variances (with p=0.01). Using this step, a total of 2676 probe sets were selected. A non paired randomized variance t-11 test with a p=0.05 (because of the small size of the samples) was used (3).

This test generated a list of 1392 differently expressed probe sets, associated with the AFP KO phenotype. As described in the Material and Methods section, a FDR (False Discovery Rate) procedure (4-5) was applied to correct the p-value for multiple comparisons and 929 probe sets showing differential expression were validated with a p=0.05. These genes define the AFP KO female signature. The extreme values were 0,203 for the lower range (Fos: FBJ osteosarcoma oncogene) and 4,464 for the upper range (Epidermal arachidonate lipoxygenase). In this list, 47 probe sets corresponding to 39 different genes (because of the redundancy of probe sets) showed a minimum fold change of 2. WT males were compared to the AFP KO males. No anomalies were detected in the AFP KO males; the male pituitaries were thus not further analyzed.

The microarray results obtained in female animals were then validated by quantitative RT-PCR experiments. Among the 929 genes showing differential expression, 34 genes were selected according to two criteria: their biological relevance in fertility pathways (GnRH receptor pathway, lactation) or in neuronal activity, and their difference in expression level, to cover a wide range of up and down-regulation in the AFP KO female pituitaries. A total of 33 out of the 34 selected genes also showed differential expression between WT and AFP KO females (the only exception was the Psa gene), thereby largely confirming the results from the microarray analysis. It is noticeable that among the genes defining the AFP KO female signature, several ones were previously reported to be important in female fertility, on the basis of the phenotype observed in the corresponding knock-out mouse models, namely: Egr1 (27), Cish2 (8), Ptprf (35), Psa (33), and Tkt (46).

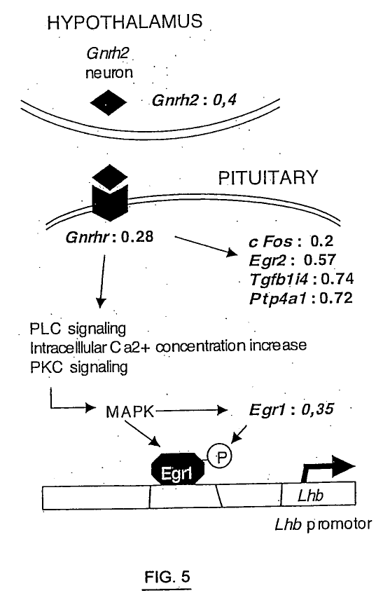

As reported previously, AFP KO females show an anovulatory phenotype (20). The GnRH receptor pathway is particularly important for ovulation, and this pathway is disturbed in the AFP KO females Indeed, the GnRH receptor mRNA level is reduced by a factor of 3.5 (microarrays) to 1.7 (quantitative RT-PCR) in the AFP KO females compared to the WT females. The FIG. 5 represents a schematic view of the GnRH pathway. Genes the expression of which is affected in the AFP KO. Females are indicated in bold. Fold changes in mRNA levels are shown as ratios AFP KO:WT.

Several downstream genes are also downregulated as described below. The GNRH receptor is a seven transmembrane domain G protein-coupled receptor. The Number of these receptor molecules varies over the estrous cycle and is thought to correlate with the gonadotropin secretory capacity of the pituitary gonadotroph cells (12). It is also correlated to the level and the frequency of release (pulsatility) of its ligand, the decapeptide GNRH produced by the hypothalamus (11, 14, 29). Binding of GNRH to its receptor induces a rise in intracellular Ca2+ concentration and activates the diacylglycerol-protein kinase C (PKC) pathway (for review 1 see 9). This pathway directly influences the Egr1 transcription factor that binds to the luteinizing hormone beta subunit gene (Lhb) promoter thereby activating its transcription. When the GNRH receptor is activated, the Egr1 mRNA level increases and the activation effect of Egr1 on the Lhb promoter is further enhanced by PKC-elicited phosphorylation (18, 41). In accordance with a down-regulation of the GnRH receptor gene in the AFP KO female pituitary, the Egr1 mRNA is down-regulated in these mice (FIG. 5). Furthermore, Fos, the mRNA level of which correlates with that of Egr1 (37), is also reduced (FIG. 5). The expression level of Fosb is also down-regulated in the AFP KO female pituitary, suggesting that both Fos and Fosb could be regulated by Egr1. In addition, differences were observed in the expression level of three other genes previously described as being part of the GNRH receptor-coupled gene network (44), namely: transforming growth factor beta 1 induced transcript 4 (Tgfb1i4), the protein tyrosine phosphatase 4a1 (Ptp4a1) and early growth response 2 (Egr2). The first two genes are down-regulated in the AFP KO females by a factor of 1.3 (FIG. 5) and Egr2 is down-regulated by a 1.7 factor. On the other hand, other hormonal secretions of the pituitary represented on the Mouse expression set 430A showed no difference of expression level between WT females and AFP KO females, namely: Gh (growth hormone), Prl (prolactin), Fsh (follicle stimulating hormone) and Pomc (pro-opiomelanocortin). Thus, differential expression of pituitary hormonal transcripts between WT and AFP KO females seems to be limited to the GNRH receptor pathway. Since the GNRH receptor level is down-regulated in the AFP KO female pituitary and since this level is known to be linked to the level of its ligand in a dose dependent manner (14), the gonadotropin-releasing hormone (Gnrh2) mRNA level was quantified in the hypothalamus (by quantitative RT-PCR with the SYBR Green dye). The Gnrh2 mRNA was found to be down-regulated by a factor. 2.5 in the AFP KO female hypothalamus (FIG. 5) (no 2) difference was found between WT and AFP KO males).

### Example 3

### Estrogen-Free Embryo Development Rescues Fertility in AFP KO Females

Fertility

AFP KO females are sterile either because AFP could not play its neuroprotective role against the effects of circulating estrogens, or because AFP could not actively bring estrogens to specific brain cells. In order to discriminate between these two hypotheses, the aromatase inhibitor ATD was injected to block estrogen synthesis in heterozygous females during the late gestational period, which is a critical time period for brain sexual differentiation. The former hypothesis predicts that AFP KO female fetuses which developed in an estrogen-free environment should be fertile (no estrogen, no need for protective AFP). On the contrary, the latter hypothesis predicts that AFP KO females should be sterile (no estrogen-carrier AFP). Serum estradiol levels of the ATD-treated gestating females proved the effectiveness of the treatment (estradiol levels are below the 10 pg/ml detection limit of the RIA assay).

Heterozygous gestating females (13 animals) were injected with ATD and a total of 17 AFP KO female pups and 25 heterozygous female pups were retrieved. The fertility of 8 of the AFP KO pups and of 17 of the heterozygous pups was tested: females of both genotypes were fertile and gave birth to litters of normal size. Furthermore, 3 of these AFP KO females and 5 of the heterozygous ones were then mated again to determine whether additional litters could be obtained. Both genotypes were fertile again and gave birth to litters of normal size comprising both sterile AFP KO and fertile heterozygous females. The correction of the sterility of the treated AFP KO females was thus not transmitted, as expected. As controls, heterozygous females were injected with propylene glycol only (no ATD). They gave birth to sterile AFP KO and fertile heterozygous females.

Gene Expression

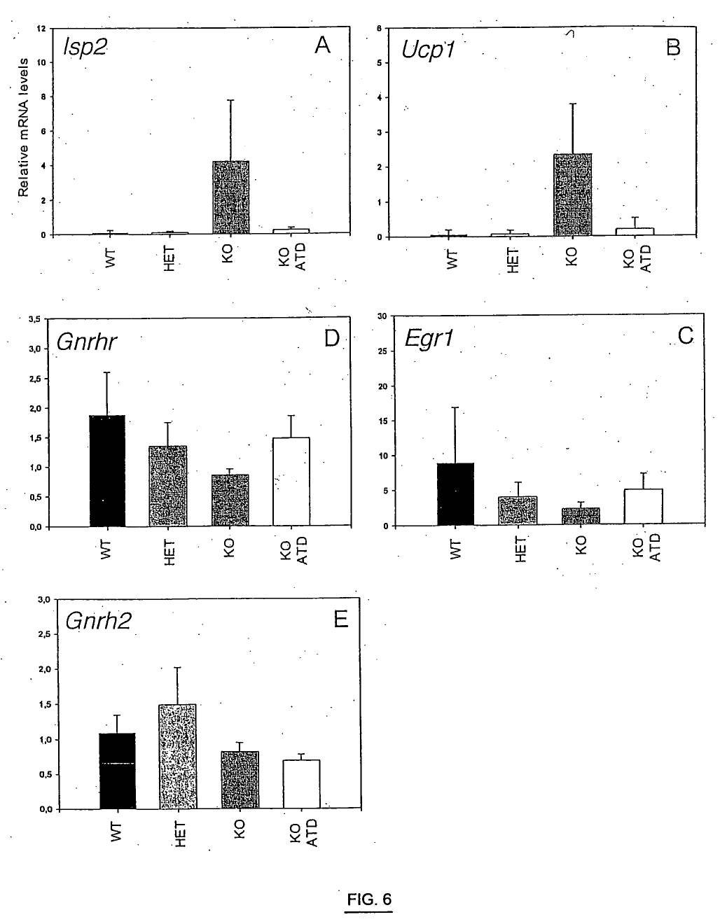

Since prenatal ATD treatment restored the fertility of the AFP KO females, this treatment could also normalize the gene expression level in the hypothalamo-pituitary axis of these mice. The expression of several genes was tested in the pituitary, i.e. Ucp1 (uncoupling protein 1), Isp2 (implantation serine protease 2), Egr1 (early growth response 1) and Gnrhr (GnRH receptor). The results are shown in FIG. 6.

The FIG. 6 represents relative expression levels of genes in the pituitary (Isp2, Ucp1, Egr1, Gnrhr) and the hypothalamus (Gnrh2). Values are normalized with the Hprt gene values. WT: wild type allele homozygous females (n=16); HET: heterozygous females (n=9); KO: homozygous AFP KO females (n=7); KO ATD: prenatally ATD-treated AFP KO females (n=7). KO-ATD KO differences in the pituitary are significant (p=0.044) while HET-ATD KO differences are not (p=0.508), except for Isp2 (p=0.014). For Gnrh2, WT-HET differences are not significant, while KO-KO ATD and KO ATD-HET are significant (p=0.049 and p=0.002 respectively).

Wild type and heterozygous animals were different from those used for microarray analysis but were of the same back-cross level as the ATD treated and untreated AFP KO mice. Prenatal ATD-treated AFP KO females exhibited expression levels that were statistically different from those of the untreated KO females (p=0.044), but not statistically different from those of the heterozygous, untreated females (p=0.508) except for the Isp2 gene in which prenatal ATD treated KO females are statistically different from heterozygous untreated females. The expression of these genes reached thus expression levels of fertile groups again. The Gnrh2 gene expression in the hypothalamus was tested. Surprisingly, the Gnrh2 expression level in the prenatal ATD-treated AFP KO mice was not increased (FIG. 6).

AP is involved in female fertility (20) and taking into account this result, a critical property of AFP is its capacity to bind estrogens (6, 32, 34, 43). Whether this binding reflects a passive, neuroprotective role or a carrier activity remained a matter of debate (17, 40). The AFP KO mouse allows us to clarify this issue. Fertility of the AFP KO mice is restored after development in an estrogen-free environment. This result allows the conclusion that AFP has a mere passive, neuroprotective role. However, the fact that aromatase knock-out females mice are sterile (19) and remain unable to ovulate even after adult estradiol treatment (39) points out that more than prenatal estrogen shielding of the brain alone is needed to achieve normal female reproductive function in adulthood, and that estrogen exposure in the postnatal period is necessary for further sexual differentiation.

This study shows that prenatal overexposure to estrogen, as a result of absence of embryonic AFP, affects the developing brain and results in anomalies in the level of numerous gene transcripts in adulthood. In particular, the GnRH pathway is downregulated. These results are in accordance with in vitro studies showing that cellular differentiation and migration of cultured GnRH cells are inhibited if the cultures are exposed to AFP antibodies (16). The fact that this pathway is stably disturbed while that the other main hormonal secretions of the pituitary are unaffected is in accordance with the phenotype of the AFP KO mice in which anovulation is the only phenotypic anomaly detected. Correct integration of the hypothalamic GnRH surge through its pituitary receptor is responsible for the preovulatory LH surge and ultimately for ovulation. The Egr1 knock-out females share many phenotypic similarities with the AFP KO females. EGR1 is a transcription factor that binds the Lhb promotor and is downregulated in the AFP KO female mice. Lhb mRNA levels are not affected in the female AFP KO mice but since these mice do not cycle properly, they could not be analyzed in the proestrus phase of the sexual cycle in which the LH surge occurs and consequently, the Lhb mRNA levels measured are basal levels, which thus appear to be normal.

AFP KO male mice show no differences in Gnrh2, Gnrhr or Fsh gene expression, which is consistent with their normal phenotype. Thus, the absence of embryonic AFP seems to interfere with female brain development only.

Prenatal treatment of AFP KO female mice with an aromatase inhibitor not only rescues fertility, but also restores the expression profile of the tested genes in the pituitary to values similar to those of fertile heterozygous females. Interestingly, heterozygous females, while fertile, show a GnRH receptor gene expression level intermediate between those of wild type and AFP KO females, pointing out an AFP dose dependent effect. Unexpectedly, in the hypothalamus, the expression of the Gnrh2 gene remains abnormally low, while the level of the pituitary Gnrhr mRNA is normalized. As Gnrh2 is a highly regulated gene, with transcriptional and post-transcriptional regulations, it is possible that stabilization of the decapeptide occurs, compensating the low mRNA level. GnRH is capable of regulating its own secretion by ultrashort feed-back mechanisms mediated by GnRH receptors present in a subpopulation of GnRH neurons (45). On the other hand, since pulsatility of the GnRH action is a critical feature (13), another explanation might be that in ATD treated AFP KO females, even suboptimal levels of GnRH could elicit an adequate pituitary response provided they are delivered in the right pulsatile manner. Anovulation caused by a dysfunction of the hypothalamo-pituitary axis is frequently observed in women consulting for fertility issues.

### REFERENCES

1). Abelev, G. I. et al. 1967 Int. J. Cancer. 2:551-558.
2). Andrews, G. K., et al. 1982 J. Biol. Chem. 257:5148-5153.
3). Baldi, P., and A. D. Long. 2001 Bioinformatics 17:509-519.
4). Benjamini, Y., and Yekutieli, D. 2001 Ann. Stat. 29:1165-1188.
5). Benjamini, Y., and Y. Hochberg. 1995 J. Roy. Stat. Soc. B. Met. 57:289-300.
6). Bennett, J. A., et al. 2002 PNAS USA 99: 2211-2215.
7). Brand, T., et al. 1991 Horm. Behav. 25:323-341.
8). Cargill, S. L, J. F. Medrano, and G. B. Anderson 1999 Biol. Reprod. 61:283-287.
9). Cheng, K. W., and P. C. Leung 2000 Can. J. Physiol.Pharmacol. 78:1029-1052.
10). Chiu, J. F., et al. 1983 Arch Biochem Biophys. 222:310-320.
11). Clayton, R. N. 1988 Hum. Reprod. 3:479-483.
12). Clayton, R. N., et al. 1980 Endocrinology. 107:699-706.
13). Conn, P. M., et al. 1984 Pharmacol. 25:51-55.
14). Conn, P. M., and W. F. Crowley Jr. 1994 Annu. Rev. Med. 45:391-405.
15). Crowley, W. R., and S. P. Kalra. 1994 Brain Res. 663:257-265.
16). Daikoku, S., and I. Koide 1994 Horm. Behav. 28:328-335.
17). Dohler K D. 1991 Int. Rev. Cytol. 131:1-57.
18). Duan, W. R., et al. 2002 Mol. Endocrinol. 16:221-233.
19). Fisher, C. R., et al. 1998 PNAS USA 95:6965-6970.
20). Gabant, P., et al. 2002 PNAS USA 99:12865-12870.
21). Gore, A. C., and J. L. Roberts 1997 Front. Neuroendocrinol. 18:209-245.
22) Gorski, R. A. 1985 J. Anim. Sci. 61:38-61.
23). Irizarry, R. A. et al. 2003 Biostatistics. 4:249-264.
24). Irizarry, R. A., L. Gautier, L. Cope 2003 An R package for oligonucleotide array statistical analysis. pp. 455-450. In Parmigiani, G., Garrett, E. S., Irizarry, R. and Zeger, S. L. (eds), The Analysis of Gene Expression Data: Methods and Software. Springer, New York, N.Y.
25). Jones, E. A. et al. 2001 J. Anat. 198:555-559.
26). Labdenne, P., and M. Heikinheimo 2002 Ann. Med. 34:316-323.
27). Milbrandt 1996 Science. 273:1219-1221.
28). MacLusky, N. J. and F. Naftolin 1981 Science 211:1294-1302.
29). Marian, J., R. L. Cooper, and P. M. Conn 1981 Pharmacol. 19:399-405.
30). Masopust, J., K. et al. 1968 Int. J. Cancer. 3:364-373.
31). Mizejewski, G. J. 1985 In: Mizejewski G J, Porter I H, Eds. Alha-fetoprotein and Congenital Disorders—Orlando: Academic Press, pp 5-34.
32). Nishi, S. et al. 1991 PNAS USA 88 :3102-3105.
33). Osada, T. et al. 2001 Mol. Endocrinol. 15:960-971.
34). Savu, L. et al. Nunez. 1981 J. Biol. Chem. 256:9414-9418.
35). Schaapveld, R. Q. et al. Dev. Biol. 188:134-146.
36). Sharony, R. et al. 2004 Eur. J. Hum. Genet. 12:871-874.
37). Sukhatme V. P. et al. 1988 Cell. 53:37-43.
38). Tilghman, S. M., and A. Belayew 1982 PNAS USA 79:5254-5257.
39). Toda, K. et al. 2001 J. Endocrinol. 170:99-111.
40). Toran-Allerand, C. D. 1984 Prog. Brain. Res. 61:63-98.
41). Tremblay, & J. J. Drouin 1999 Mol. Cell. Biol. 19:2567-2576.
42). Uriel, J., D. et al. 1976 PNAS USA 73:1452-1456.
43). Vakharia, D. & G. J. Mizejewski 2000 Breast Cancer Res. Treat. 63:41-52
44) Wurmbach, E. et al. 2001 J. Biol. Chem. 276:47195-47201.
45). Xu, C. et al. 2004 Endocrinology. 145:728-735.
46). Xu, Z. P. et al. 2002 Mol. Cell. Biol. 22:6142-6147.
47). Tilghman S. M. & Belayew A. 1982 PNAS USA 79:5254-5257.
48). Godbout R. et al. 1986 Mol. Cell. Biol. 6:477-487.
49). Godbout R. et al. 1998 Mol. Cell. Biol. 8:1169-1178.
50). Chen H. et al. 1997 Critical Rev. In Eucaryotic Gene Expression 7:11-41.
51). Festin et al. 1999 Biochem. & Biophys. Acta 24789:307-314.
52). Leffert H. L. and Sell S. 1974 J. Cell Biol. 61:823-829.
53). Sell S. et al. 1975 Ann. NY Acad. Sci. 259: 45-58.
54). Tsukada Y. & Hirai H. 1975 Ann. NY Acad. Sci. 259:37-44.
55). Belanger L. et al. 1978 Scand. J. Immunol. 8:239-246.
56). Mizejewski G. J. 1985 New insights into AFP structure and function: potential biomedical applications. In Mizejewski G. J., Porter I H (eds). Alpha-Fetoprotein and congenital disorder. Orlando:Academic Press, 5-34.
57). Mizejewski G. J. 1995 Life Sci. 56:1-9.

58). Abe K. et al. 1996 Exp. Cell Res. 25:27-34.

TABLE 4
Selected genes differently expressed in the female AFP KO pituitaries.

| | | | FCGM as | |
|---|---|---|---|---|
| | | | established by | |
| Affymetrix | Unigene | Gene | | RT- |
| probe set | number | symbol | Microarray | PCR |

A. Genes differentially expressed by a factor ≧2 (microarray analysis)

| | | | | |
|---|---|---|---|---|
| 1423100_at | Mm.246513 | Fos | 0.203 | 0.462 |
| 1417025_at | Mm.22564 | H2-Eb1 | 0.277 | |
| 1450407_a_at | Mm.269088 | Anp32a | 0.278 | |
| 1418188_a_at | Mm.358667 | Ramp2 | 0.281 | |
| 1421665_a_at | Mm.26791 | Gnrhr | 0.285 | 0.588 |
| 1416236_a_at | Mm.33240 | Eva1 | 0.310 | 0.535 |
| 1424609_a_at | Mm.182434 | Fstl1 | 0.317 | |
| 1448107_x_at | Mm.142722 | Klk6 | 0.327 | |
| 1424607_a_at | Mm.181959 | Egr1 | 0.348 | 0.628 |
| 1456341_a_at | Mm.291595 | Bteb1 | 0.351 | |
| 1427104_at | Mm.87487 | Zfp612 | 0.378 | |
| 1438676_at | Mm.275893 | Mpa21 | 0.391 | |
| 1422809_at | Mm.309296 | Rims2 | 0.400 | 0.552 |
| 1454607_s_at | Mm.289936 | Psat1 | 0.405 | |
| 1455988_a_at | Mm.153159 | Cct6a | 0.407 | |
| 1448830_at | Mm.239041 | Dusp1 | 0.409 | |
| 1417513_at | Mm.35796 | Evi5 | 0.413 | |
| 1418908_at | Mm.5121 | Pam | 0.423 | |
| 1423804_a_at | Mm.29847 | Idi1 | 0.426 | |
| 1437082_at | Mm.46044 | Akap9 | 0.442 | |
| 1423619_at | Mm.3903 | Rasd1 | 0.449 | |
| 1456175_a_at | Mm.261735 | Copb2 | 0.454 | |
| 1416131_s_at | Mm.260647 | C920006C10Rik | 0.462 | |
| 1434113_a_at | Mm.252772 | Xpmc2h | 0.472 | |
| 1429296_at | Mm.74596 | Rab10 | 0.475 | |
| 1426956_a_at | Mm.215389 | Trp53bp1 | 0.476 | |
| 1452377_at | Mm.2389 | M$$ | 0.478 | |
| 1434578_x_at | Mm.297440 | Ran | 0.485 | |
| 1433446_at | Mm.61526 | Hmgcs1 | 0.490 | 0.593 |
| 1434380_at | Mm.254851 | Dnr12 | 0.498 | |
| 1455141_at | Mm.218873 | Tnrc6a | 0.500 | |
| 1448312_at | Mm.294493 | Pcsk2 | 2.012 | 1.729 |
| 1420575_at | Mm.2064 | Mt3 | 2.141 | 1.521 |
| 1428014_at | Mm.158272 | Hist1h4h | 2.252 | |
| 1449992_at | Mm.207082 | Isp2-pending | 2.611 | 35.750 |
| 1430111_a_at | Mm.4606 | Bcat1 | 2.924 | |
| 1418197_at | Mm.4177 | Ucp1 | 3.049 | 132.265 |
| 1420338_at | Mm.4584 | Alox15 | 3.067 | 75.309 |
| 1426039_a_at | Mm.274093 | Alox12c | 4.464 | 31.213 |

B. Additional genes tested by quantitative RT-PCR.

| | | | | |
|---|---|---|---|---|
| 1450033_a_at | Mm.277406 | Stat1 | 0.554 | 0.398 |
| 1422629_s_at | Mm.46014 | Shrm | 0.563 | 0.546 |
| 1451718_at | Mm.1268 | Plp | 0.567 | 0.342 |
| 1427683_at | Mm.290421 | Egr2 | 0.570 | 0.620 |
| 1424470_a_at | Mm.24028 | 9330170P05Rik | 0.592 | 0.607 |
| 1450646_at | Mm.140158 | Cyp51 | 0.606 | 0.655 |
| 1421191_s_at | Mm.142822 | Gopc | 0.634 | 0.636 |
| 1452036_a_at | Mm.159684 | Tmpo | 0.643 | 0.646 |
| 1417385_at | Mm.29824 | Psa | 0.652 | 0.905 |
| 1426819_at | Mm.248335 | Fosb | 0.658 | 0.464 |
| 1460248_at | Mm.10233 | Cpxm2 | 0.663 | 0.589 |
| 1417444_at | Mm.153415 | E2f5 | 0.714 | 0.644 |
| 1460329_at | Mm.26364 | B4galt6 | 0.756 | 0.642 |
| 1448754_at | Mm.279741 | Rbp1 | 0.769 | 0.462 |
| 1451695_a_at | Mm.332810 | Gpx4 | 1.391 | 1.483 |
| 1419469_at | Mm.139192 | Gnb4 | 1.499 | 1.519 |
| 1424171_a_at | Mm.43784 | Hagb | 1.543 | 1.499 |
| 1417111_at | Mm.117294 | Man1a | 1.546 | 2.540 |
| 1417502_at | Mm.18590 | Tm4sf2 | 1.590 | 2.192 |
| 1422557_s_at | Mm.192991 | Mt1 | 1.645 | 1.554 |
| 1422586_at | Mm.140765 | Ecel1 | 1.815 | 2.948 |
| 1451342_at | Mm.334160 | Spon1 | 1.946 | 5.139 |

What is claimed is:

1. A diagnostic kit to identify if a female mammal subject comprises a mutation, partial or total deletion of alpha feto-protein (AFP DNA sequence) in her genome and which comprises specific sequences that are able to hybridize specifically with the alpha-fetoprotein DNA sequence and/or amplify at least a portion of the alpha-fetoprotein DNA sequence.

2. The diagnostic kit according to the claim 1, wherein the specific sequences are primers.

3. The diagnostic kit according to the claim 1, wherein the specific sequences are probes.

4. The diagnostic kit according to claim 1, wherein the sequences are bound to a solid support according to a microarray.

5. The diagnostic kit according to claim 1, wherein the specific sequences are able to hybridize specifically with the AFP domain III and/or amplify the AFP domain III.

6. The diagnostic kit according to claim 1, further comprising means and media for a detection of hybridized sequences or amplified sequences by a method selected from the group consisting of fluorescence detection, chemoluminescence detection, bioluminescence detection, colorimetric detection and radioactive labeling detection.

7. A method to identify if a female mammal subject (including a female human patient) comprises a mutation, partial or total deletion of the alpha-fetoprotein (AFP) DNA sequence in her genome, which comprises the steps of:
putting into contact a biological sample obtained from the mammal subject, said biological sample comprising the genome of the mammal subject, with sequences that are able to hybridize specifically with the alpha-fetoprotein DNA sequence and/or amplify at least a portion of the alpha-fetoprotein DNA sequence;
detecting these hybridized sequences or these amplified sequences and detecting if these DNA sequences present a mutation, partial or total deletion.

8. The method according to the claim 7, wherein the phenotype of the mammal subject is selected from the group consisting of female mammal sterility, no menstrual cyclization and/or no possible uteral implantation of an embryo.

9. The method according to the claim 7, further comprising correlating said mutation, partial or total deletion to a phenotype of the mammal subject.

10. A method of treatment and/or prevention of a phenotype selected from the group consisting of female sterility, no menstrual cyclization and/or no uteral implantation of an embryo expressed by a female mammal subject (including a female human patient) and which comprises the steps of:
identifying if a mammal subject present a mutation, partial or total deletion in the alpha-fetoprotein DNA sequence in its genome and may transmit said mutation, partial or total deletion to its descendant(s), (especially to her daughter) that may present said mutation, partial or total deletion homozygously and associated with the said phenotype, and
treating the mammal by hormone therapy during gestation, in order to allow that the female mammal gives birth to a female mammal which does not present the phenotype associated with the mutation, or the partial or total deletion of the AFP DNA sequence.

11. The method according to the claim 10, wherein the treatment of the mammal comprises administration of one or more aromatase inhibitors.
